(19) United States
(12) Patent Application Publication
(10) Pub. No.: US 2013/0137974 A1
(43) Pub. Date: May 30, 2013

(54) X-RAY IMAGING APPARATUS, MEDICAL IMAGE PROCESSING APPARATUS AND X-RAY IMAGING METHOD

(71) Applicants: Takuya SAKAGUCHI, Utsunomiya-shi (JP); Kyojiro NAMBU, Nasushiobara-shi (JP); Atsushi GOTOH, Irvine, CA (US); Ryuji ZAIKI, Utsunomiya-shi (JP)

(72) Inventors: Takuya SAKAGUCHI, Utsunomiya-shi (JP); Kyojiro NAMBU, Nasushiobara-shi (JP); Atsushi GOTOH, Irvine, CA (US); Ryuji ZAIKI, Utsunomiya-shi (JP)

(21) Appl. No.: 13/685,093

(22) Filed: Nov. 26, 2012

(30) Foreign Application Priority Data
Nov. 29, 2011 (JP) 2011-261020
Sep. 19, 2012 (JP) 2012-205527

(51) Int. Cl. G03B 42/02; A61B 5/00

(52) U.S. Cl. CPC G03B 42/026; A61B 5/0044
USPC 600/424; 378/98.2

(57) ABSTRACT

According to one embodiment, an X-ray imaging apparatus includes an angle acquisition unit and an imaging unit. The angle acquisition unit is configured to acquire an inclined angle of an aorta of an object on a reference image. The imaging unit is configured to display an X-ray image with rotating an angle so as to make a travel direction of the aorta to be a horizontal direction, a vertical direction or a predetermined direction corresponding to a craft. The angle is rotated based on the inclined angle of the aorta.

### CROSS REFERENCE TO RELATED APPLICATION

This application is based upon and claims the benefit of priority from Japanese Patent Application No. 2011-261020, filed Nov. 29, 2011 and Japanese Patent Application No. 2012-205527, filed Sep. 19, 2012; the entire contents of which are incorporated herein by reference.

### FIELD

Embodiments described herein relate generally to an X-ray imaging apparatus, a medical image processing apparatus and an X-ray imaging method.

### BACKGROUND

Conventionally, a technique for an intervention treatment in real time with observing images of a body of an object obtained by an X-ray imaging apparatus is known. For example, a device such as a catheter, a guide wire, a stent, a stent graft, and an artificial valve can be placed in a body of an object through a tube inserted in a blood vessel.

The replacement of an aortic valve is one of the treatments placing a device in a body. The replacement of an aortic valve is a treatment technique placing an artificial valve in an aorta through a catheter inserted from a blood vessel of a femur. The replacement technique of an aortic valve using a catheter is called as the TAVR (Trans-catheter Aortic Valve Replacement) or TAVI (Trans-catheter Aortic Valve Implantation).

It is essential for the replacement technique of an aortic valve to place an artificial valve at an appropriate position so that the artificial valve becomes perpendicular to the aorta. This is because the artificial valve does not spread properly and the treatment purpose does not be achieved if the artificial valve placed obliquely to the aorta.

However, a doctor conventionally performs the positioning of the artificial valve by observing visually fluoroscopic images of the aorta and the valve depicted in an oblique direction on a screen. Specifically, the shapes and the positions of the valve and the aorta are confirmed by the visual observation with contrast-enhancing of the blood vessel when the artificial valve reaches the vicinity of the natural valve.

Therefore, it is difficult to perform the positioning of the artificial valve and there is a possibility that the artificial valve may be placed obliquely. Furthermore, there is a problem that it is difficult to recognize the artificial valve has placed slightly obliquely.

Accordingly, it is an object of the present invention to provide an X-ray imaging apparatus, a medical image processing apparatus and an X-ray imaging method which make it possible to assist placing an artificial valve in the appropriate direction in the replacement technique of an aortic valve.

### BRIEF DESCRIPTION OF THE DRAWINGS

In the accompanying drawings:

FIG. 1 is a configuration diagram of an X-ray imaging apparatus and a medical image processing apparatus according to the first embodiment of the present invention;

FIG. 2 shows an example of X-ray image data of a heart, depicting an aorta, generated for a reference of a replacement for an aortic valve, in the image generation part;

FIG. 3 shows an example of a balloon image to be a target for automatic detection of an aortic inclined angle by the first angle acquisition part shown in FIG. 1;

FIG. 4 shows an example of figure for instructing the inclined angle, overlapped and displayed on an X-ray image depicting the aorta, by the first angle acquisition part shown in FIG. 1;

FIG. 5 shows an example of X-ray image, displayed and rotated so that the aorta becomes the vertical direction, and ROI set again;

FIG. 6 shows an example of marks displayed for visually recognizing the horizontal direction and the vertical direction on an X-ray image displayed and rotated so that the aorta becomes the vertical direction;

FIG. 7 is a diagram describing a method for translating an X-ray image using the marks displayed at fixed positions on the screen;

FIG. 8 is a flowchart showing a flow for displaying X-ray images for a replacement of an aortic valve of an object by the X-ray imaging apparatus shown in FIG. 1; and

FIG. 9 is a view for explaining functions of an X-ray imaging apparatus and a medical image processing apparatus according to the second embodiment of the present invention.

### DETAILED DESCRIPTION

In general, according to one embodiment, an X-ray imaging apparatus includes an angle acquisition unit and an imaging unit. The angle acquisition unit is configured to acquire an inclined angle of an aorta of an object on a reference image. The imaging unit is configured to display an X-ray image with rotating an angle so as to make a travel direction of the aorta to be a horizontal direction, a vertical direction or a predetermined direction corresponding to a craft. The angle is rotated based on the inclined angle of the aorta.

Further, according to another embodiment, a medical image processing apparatus includes an angle acquisition unit and a display control unit. The angle acquisition unit is configured to acquire an inclined angle of an aorta of an object on a reference image. The display control unit is configured to display an X-ray image with rotating an angle so as to make a travel direction of the aorta to be a horizontal direction, a vertical direction or a predetermined direction corresponding to a craft. The angle is rotated based on the inclined angle of the aorta.

Further, according to another embodiment, an X-ray imaging method includes acquiring an inclined angle of an aorta of an object on a reference image; and displaying an X-ray image with rotating an angle so as to make a travel direction of the aorta to be a horizontal direction, a vertical direction or a predetermined direction corresponding to a craft. The angle is rotated based on the inclined angle of the aorta.

An X-ray imaging apparatus, a medical image processing apparatus and an X-ray imaging method according to embodiments of the present invention will be described with reference to the accompanying drawings.

### First Embodiment

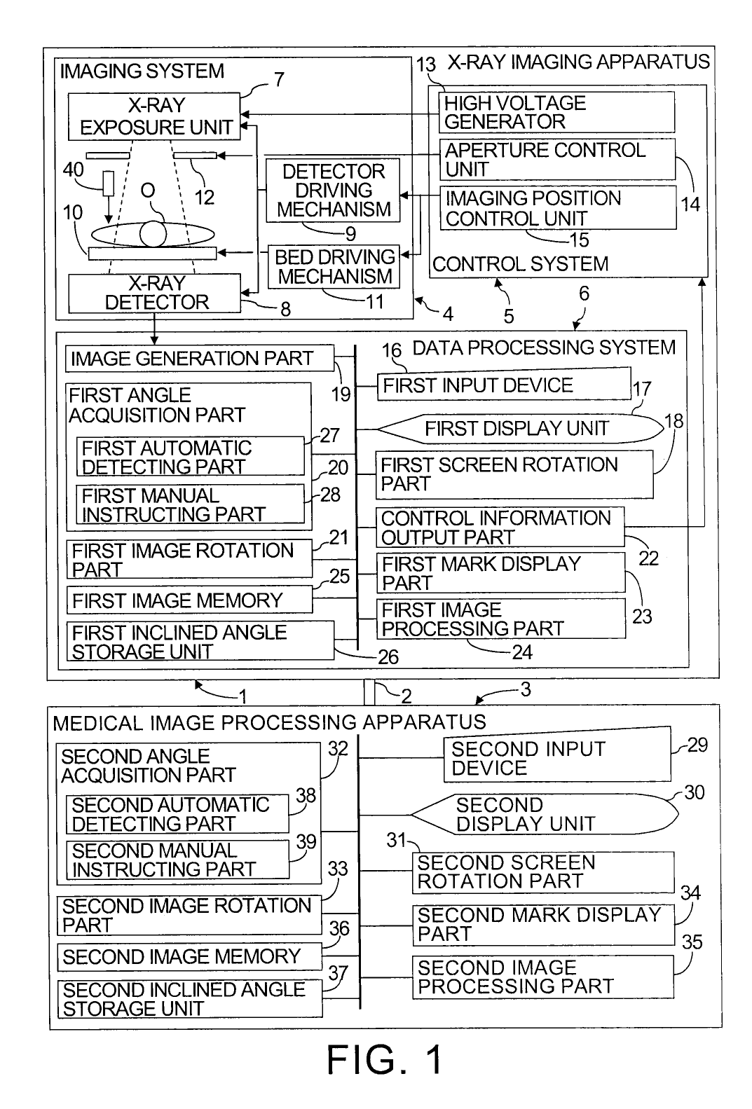

FIG. 1 is a configuration diagram of an X-ray imaging apparatus and a medical image processing apparatus according to the first embodiment of the present invention.

An X-ray imaging apparatus 1 is connected to a medical image processing apparatus 3 through a network 2. Then, they are configured to transmit X-ray medical image data obtained in the X-ray imaging apparatus 1 to the medical image processing apparatus 3 through the network 2 and perform desired image processing of the X-ray medical image data in the medical image processing apparatus 3.

The X-ray imaging apparatus 1 includes an imaging system 4, a control system 5 and a data processing system 6. The imaging system 4 has an X-ray exposure unit 7, an X-ray detector 8, a detector driving mechanism 9, a bed 10, a bed driving mechanism 11 and an aperture 12. The control system 5 has a high voltage generator 13, an aperture control unit 14 and an imaging position control unit 15. The data processing system 6 has the first input device 16, the first display unit 17, the first screen rotation part 18, an image generation part 19, the first angle acquisition part 20, the first image rotation part 21, a control information output part 22, the first mark display part 23, the first image processing part 24, the first image memory 25 and the first inclined angle storage unit 26. The first angle acquisition part 20 has the first automatic detecting part 27 and the first manual instructing part 28.

On the other hand, the medical image processing apparatus 3 has the second input device 29, the second display unit 30, the second screen rotation part 31, the second angle acquisition part 32, the second image rotation part 33, the second mark display part 34, the second image processing part 35, the second image memory 36 and the second inclined angle storage unit 37. The second angle acquisition part 32 has the second automatic detecting part 38 and the second manual instructing part 39.

The elements of the data processing system 6, such as the image generation part 19, the first angle acquisition part 20, the first image rotation part 21, the first mark display part 23 and the first image processing part 24, to perform data processing can be configured by installing program in an operation unit of a computer. The elements of the medical image processing apparatus 3, such as the second angle acquisition part 32, the second image rotation part 33, the second mark display part 34 and the second image processing part 35, to perform image processing can be also configured by installing program in an operation unit of a computer. The medical image processing apparatus 3 is configured by a work station in many cases since image processing in the medical image processing apparatus 3 requires a large amount of data processing. However, circuits may be used to configure the data processing system 6 and the medical image processing apparatus 3.

The X-ray exposure unit 7 includes an X-ray tube and is placed in the opposite side of the X-ray detector 8 so that an object O set on the bed 10 lies between the X-ray exposure unit 7 and the X-ray detector 8. The X-ray exposure unit 7 and the X-ray detector 8 can change the angles and the relative positions with respect to the object O with keeping their relative position by driving the detector driving mechanism 9. Specifically, the X-ray exposure unit 7 and the X-ray detector 8 are settled at both ends of the C-shaped aim having the rotational function.

Then, the X-ray exposure unit 7 is configured to expose an X-ray from a predetermined angle to an object O by the X-ray tube to detect the X-ray transmitted the object O by the X-ray detector 8. Furthermore, the aperture 12 for adjusting the diameter of an exposed X-ray beam is placed in the output side of the X-ray tube.

On the other hand, the bed 10 includes the bed driving mechanism 11 that changes the incline and the position of the table of the bed 10. Therefore, the radiation direction of an X-ray toward an object O can be changed by adjusting not only the angles of the X-ray exposure unit 7 and the X-ray detector 8 with regard to the object O but also the angle of the table. Furthermore, a contrast agent injector 40 is provided in the vicinity of the object O set on the bed 10 in order to inject a contrast agent into the object O.

The high voltage generator 13 of the control system 5 has the function to apply a high voltage to the X-ray tube of the X-ray exposure unit 7 to expose an X-ray having a desired energy toward an object O.

The imaging position control unit 15 is the unit to control the detector driving mechanism 9 and the bed driving mechanism 11 by outputting control signals to the detector driving mechanism 9 and the bed driving mechanism 11. Specifically, the rotation angle and the position of the X-ray exposure unit 7 and the X-ray detector 8, which is adjusted by the driving of the detector driving mechanism 9, are controlled by the control signal output from the imaging position control unit 15 to the detector driving mechanism 9. Furthermore, the incline and the position of the table of the bed 10 which is adjusted by the driving of the bed driving mechanism 11 are controlled by the control signal output from the imaging position control unit 15 to the bed driving mechanism 11.

The aperture control unit 14 is the unit to control the aperture 12 by outputting the control signal to the aperture 12. Specifically, the opening of the aperture 12 is controlled according to the control signal output from the aperture control unit 14 to the aperture 12.

The first screen rotation part 18 of the data processing system 6 has the function to incline the angle of the screen of the first display unit 17, which is included in the X-ray imaging apparatus 1, on a plane parallel to the screen. Note that, in the case where the first display unit 17 has the function to incline the display angle of the screen, the screen can be inclined by using the function to incline the display screen. On the contrary, the first display unit 17 itself may be inclined mechanically in the case where the first display unit 17 does not have the function to incline the display angle of the screen. In the former case, the first screen rotation part 18 is a control unit to control the display parameters of the first display unit 17. In the latter case, the first screen rotation part 18 is a placing table having an inclining mechanism of the first display unit 17

The image generation part 19 has the functions to generate X-ray image data by acquiring X-ray detection data from the X-ray detector 8 and performing data processing of the X-ray detection data, and to display the generated X-ray image data on the first display unit 17. Especially, the image generation part 19 can generate X-ray image data that are necessary or useful for the replacement of an aortic valve.

Concrete examples include X-ray fluoroscopic image data, DSA (digital subtraction angiography) image data, road map image data, blood vessel contrast-enhanced image data and device image data, each involving a placed position of an artificial valve. The DSA image data is subtraction image data between frames of X-ray image data before and after injection of a contrast agent. The road map image data is blood vessel image data generated as composite image data of contrast-enhanced image data and fluoroscopic image data of the blood vessel in order to lead a catheter to a target position. Furthermore, the device image data is X-ray image data like balloon image data or wire image data depicting a device such as a balloon or a wire.

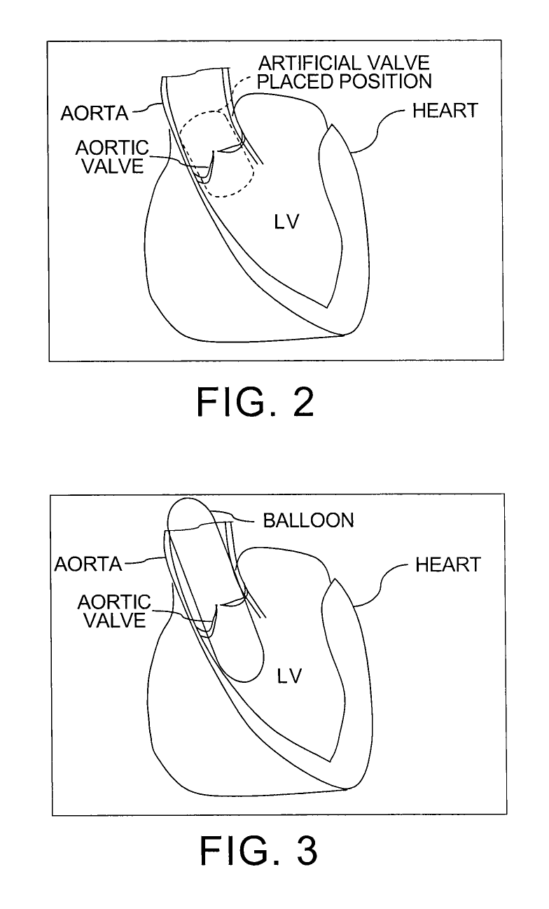

FIG. 2 shows an example of X-ray image data of a heart, depicting an aorta, generated for a reference of a replacement for an aortic valve, in the image generation part 19.

As shown in the FIG. 2, the aortic valve lies in the outflow path of the blood from the LV (LV: left ventricle) of the heart to the aorta. The artificial valve is placed at the position of the natural aortic valve shown by the dotted line in the replacement technique of the aortic valve. Accordingly, the artificial valve is placed with reference to X-ray images as shown in the FIG. 2 in advance. However, the aorta and the aortic valve in the vicinity of the LV are depicted in an oblique direction because they are inclined with respect to the body axis direction of an object O.

The first angle acquisition part 20 has the function to acquire an inclined angle of the aorta in the vicinity of the aortic valve of an object O on a reference image, with using X-ray image data, depicting at least one of the aorta of the object O and a device to confirm a position of the aorta, as reference image data. In addition, the first angle acquisition part 20 has the function to adjust the inclined angle of the aorta according to instructing information input from the first input device 16. The first automatic detecting part 27 has the function to detect the angle of the aorta automatically by image processing of the reference data. The first manual instructing part 28 has the function to overlap and display a graphic symbol, for instructing the inclined angle of the aorta, on an X-ray image displayed as a reference image on the first display unit 17; and to acquire the inclined angle of the aorta based on the rotation angle of the graphic symbol input from the first input device 16.

FIG. 3 shows an example of a balloon image to be a target for automatic detection of an aorta inclined angle by the first angle acquisition part 20 shown in FIG. 1.

As shown in the FIG. 3, a balloon inserted to the aorta and attached to the end of the catheter i s depicted on a balloon image which is one of the reference images. Accordingly, the first automatic detecting part 27 can detect the inclined angle of the aorta automatically by data processing of the balloon image data in the case where the balloon images have been acquired.

As a concrete example, an outline of a balloon can be extracted by known outline extraction processing such as subtraction processing, threshold processing, binarizing processing and edge detection processing. Then, the inclined angle of the balloon in the longitudinal direction can be obtained by calculating the inclined angle of the extracted outline in the longitudinal direction geometrically. Further, the inclined angle of the balloon can be assumed to be the inclined angle of the aorta since the balloon has been inserted in the aorta.

Similarly, it is possible to detect the inclined angle of the aorta automatically by extracting an outline of an object, whose inclined angle with respect to the aorta is known, from arbitrary reference image data to obtain the inclined angle of the extracted object. For example, a region of an object part can be extracted by removing the background components by subtraction processing between X-ray image data with the object depicted and X-ray image data without the object depicted. Further, how many degrees the object inclines to the horizontal direction or the vertical direction at about an angle of can be calculated by main component analysis of the extracted region. Alternatively, the inclined angle of the object can be also obtained by other data processing such as fitting with the known shape information of the object.

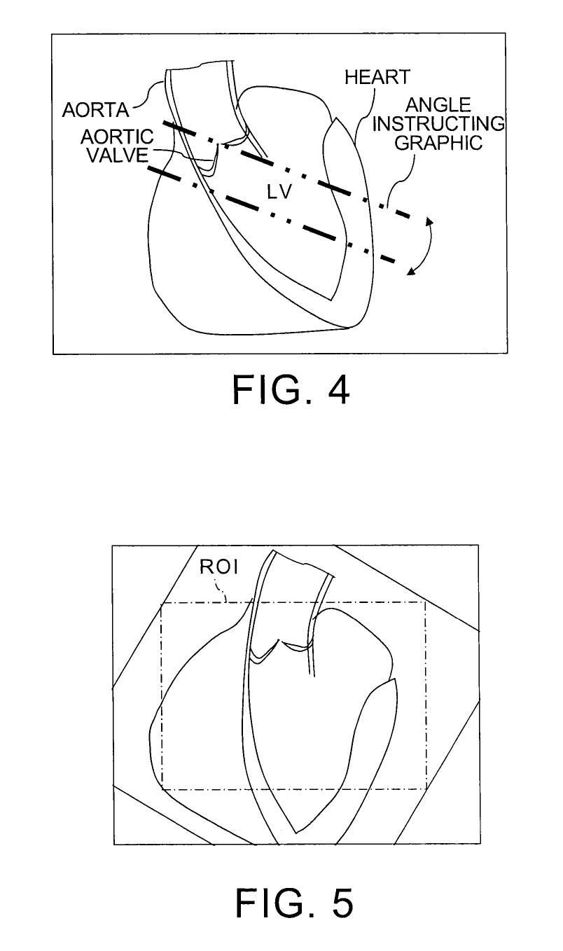

FIG. 4 shows an example of figure for instructing the inclined angle, overlapped and displayed on an X-ray image depicting the aorta, by the first angle acquisition part 20 shown in FIG. 1.

A graphic symbol to instruct the inclined angle of the aorta can be overlapped and displayed on an X-ray image depicting the aorta as shown in the FIG. 4. Though two parallel inclined lines are displayed as the graphic symbol to instruct the inclined angle in the FIG. 4, a graphic symbol, such as a line or a rectangular frame, having a desired shape can be displayed for instructing the inclined angle. Furthermore, the default of the direction of the graphic symbol may be the vertical direction or the horizontal direction.

Then, a user can rotate the graphic symbol to make the inclined angle of the graphic symbol to match the inclined angle of the aorta by the operation of the first input device 16 like a mouse. Consequently, the first manual instructing part 28 can acquire the inclined angle of the aorta based on the rotation angle of the graphic symbol input from the first input device 16.

X-ray images referred to specify the inclined angle of the aorta include a 2D (two dimensional) blood vessel contrast-enhanced image, a 2D balloon image, a 2D wire image and a 2D projection image of a three dimensional fluoroscopic image.

The inclined angle of the aorta acquired in the first angle acquisition part 20 is used as the basic information to display X-ray images with rotating the angle of the travel direction of the aorta in the vicinity of the aortic valve, which is the placed position of the artificial valve, so that the travel direction of the aorta becomes the horizontal or vertical direction. The methods for rotating a displayed X-ray image include the method for rotating the X-ray detector 8, the method, the method for rotating an object O and the method, the method for rotating the display screen of the X-ray image in addition to the method for rotating the X-ray image itself by image processing.

The inclined angle of the aorta acquired in the first angle acquisition part 20 can be stored in the first inclined angle storage unit 26. Therefore, the inclined angle of the aorta can be retrieved from the first inclined angle storage unit 26 at a desired timing to be used for the rotated display of X-ray images.

The first image rotation part 21 has the function to rotate an X-ray image displayed on the first display unit 17 by image processing of X-ray imaging data involving the aorta based on the inclined angle of the aorta acquired in the first angle acquisition part 20.

Specifically, the first image rotation part 21 acquires X-ray image data output from the image generation part 19 to the first display unit 17 in the case where the information instructing the rotated display by the image processing of X-ray images has been input from the first input device 16. Then, the first image rotation part 21 generates X-ray image data of which travel direction of the aorta is in the horizontal or vertical direction by rotational processing for rotating each pixel position by an angle between the inclined angle of the aorta and the horizontal or vertical direction. Next, the first image rotation part 21 outputs the generated X-ray image data to the first display unit 17. Herewith, an X-ray image being displayed on the first display unit 17 is updated to a rotated X-ray image.

The control information output part 22 has the function to output control information, necessary for acquiring X-ray image data, to the imaging position control unit 15 and the aperture control unit 14 to control them according to instructing information from the first input device 16. Especially, the control information output part 22 has the function to control the imaging position control unit 15 or the first screen rotation part 18 based on the inclined angle of the aorta acquired in the first angle acquisition part 20 so that X-ray images, of which travel direction of the aorta is directed toward the horizontal or the vertical direction, are displayed in the first display unit 17. The control information output part 22 also has the function to control the aperture control unit 14 based on the inclined angle of the aorta so that unnecessary regions should not be displayed in case of displaying X-ray images with rotation. The control target of the control information output part 22 can be instructed by the operation of the first input device 16.

Specifically, when an instruction to tilt at least one of the X-ray detector 8 and the object O has been input to the control information output part 22 from the first input device 16, the control information output part 22 displays X-ray images, of which angle of the aorta has been rotated into the horizontal or vertical angle, on the first display unit 17 by tilting at least one of the X-ray detector 8 and the object O based on the inclined angle of the aorta acquired in the first angle acquisition part 20.

More specifically, the control information output part 22 controls the imaging position control unit 15 by outputting the control information to the imaging position control unit 15 so as to rotate the X-ray detector 8 or the table of the bed 10 by an angle between the inclined angle of the aorta and the horizontal or vertical direction. In this case, either the X-ray detector 8 and the X-ray exposure unit 7 or the table of the bed 10 is inclined by an angle corresponding to the inclined angle of the aorta, under the control of the imaging position control unit 15.

On the other hand, when the instruction to incline the display screen has been input from the first input device 16 to the control information output part 22, the control information output part 22 displays X-ray images, of which angle of the aorta has been rotated into the horizontal or vertical angle, on the first display unit 17 by inclining the display screen to display the X-ray images based on the inclined angle of the aorta acquired in the first angle acquisition part 20.

Specifically, the control information output part 22 controls the first screen rotation part 18 by outputting the control information to the first screen rotation part 18 so as to rotate the display screen of the first display unit 17 by an angle between the inclined angle of the aorta and the horizontal or vertical direction. In this case, the display screen of the first display unit 17 or the first display unit 17 itself is inclined by an angle corresponding to the inclined angle of the aorta, under the control of the first screen rotation part 18.

Note that, when X-ray images to be displayed are rotated by tilting the X-ray detector 8, the object O or the first display unit 17 itself, parts having no displayed image do not appear on the corners of the display screen. Therefore, the display screen can be used effectively.

On the other hand, when X-ray images to be displayed are rotated by image processing or inclining the display screen of the first display unit 17, parts having no displayed image appear on the corners of the display screen. Accordingly, it is preferable to scale down a ROI (region of interest) which is a region for generating X-ray images so that the parts having no displayed image do not appear.

For that reason, the control information output part 22 is configured to reset a ROI so that the parts on which no images are displayed would not appear on the display screen, and to control the aperture control unit 14 according to the reset ROI.

FIG. 5 shows an example of X-ray image, displayed and rotated so that the aorta becomes the vertical direction, and ROI set again.

As shown in the FIG. 5, when X-ray images to be displayed are rotated by image processing or inclining the display screen of the first display unit 17, parts on which no images are displayed appear on the corners of the display screen. Accordingly, a ROI, by which unnecessary regions are not be displayed as shown by the dotted frame in FIG. 5, can be reset

Then, X-ray detection data can be acquired from only a ROI under the control of the aperture control unit 14 by the control information output part 22. Specifically, a ROI can be updated appropriately by the aperture 12 working with rotated and displayed X-ray images. Herewith, unnecessary exposure of an object O can be reduced. In this case, X-ray image data in a reset ROI is generated in the image generation part 19 and X-ray images in the ROI are enlarged and displayed on the first display unit 17.

The first mark display part 23 has the function to overlap and display marks such as dots, lines, a grid, and a cross symbol at desired positions on an X-ray image rotated and displayed on the first display unit 17 according to instructing information from the first input device 16.

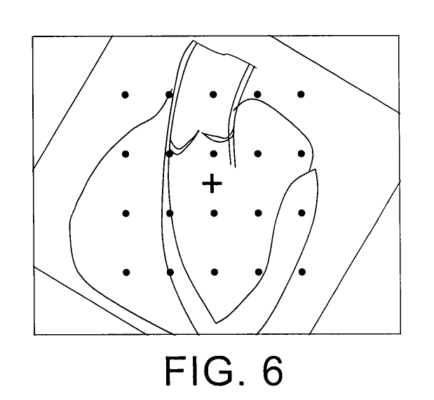

FIG. 6 shows an example of marks displayed for visually recognizing the horizontal direction and the vertical direction on an X-ray image displayed and rotated so that the aorta becomes the vertical direction.

As shown in the FIG. 6, desired marks such as dots and a cross symbol can be displayed at desired positions on a rotated and displayed X-ray image. For example, a user can confirm a distance in addition to the horizontal direction and the vertical direction visually by displaying a dot group arranged on grid points with a predetermined interval as shown in the FIG. 6. Furthermore, displaying a mark like a cross symbol at the center of the screen makes it possible to perform positioning of the artificial valve using the mark as a reference.

For example, displaying a cross symbol at the center of the screen on the first display unit 17 constantly makes it possible to make an instruction to translate a display position of an X-ray image after the rotational display by operating the first input device 16 so that the center part, the left end part or the right end part of the aortic valve lies on the cross symbol.

The mark used as a reference for adjusting the display position of the aortic valve may be not only the cross symbol as shown in FIG. 6 but also a mark shown by a line having a L-shaped, an inverse L-shaped, a rectangle or the like which is matched with the shape of the aortic valve. Specifically, a mark, matched with the shape of the aortic valve, for adjusting the display position of the aortic valve of an object can be displayed at a fixed position of the screen on the first display unit 17 on which rotated X-ray images are displayed.

For example, displaying an L-shaped mark makes it possible to give a parallel translation instruction of an X-ray image to match the left end part of the aortic valve with the L-shaped mark.

Further, the first mark display part 23 is configured to automatically adjust a shape and a size of a mark to be displayed at a fixed position on the screen of the first display unit 17, by image processing of reference image data with the aorta of the object O or a device depicted. For example, a shape of a balloon can be extracted by data processing of balloon image data generated as reference image data and the shape of the mark can be adjusted automatically based on the extracted shape of the balloon. For a more specifically example, a width of a mark of a rectangular frame to be displayed at a fixed position may be adjusted to a width of an outline of a balloon and/or a distance between mark lines shown by parallel lines may be adjusted to the width of the outline of the balloon.

Similarly, with regard to the marks, such as the dot group shown in FIG. 6 or the grid lines, to grasp a distance by visual observation, a pitch can be adjusted automatically according to an outline of a balloon or a shape of the automatically adjusted mark to be displayed at a fixed position of the screen.

Further, by inputting instructing information from the first input device 16 to the first mark display part 23, a desired mark can be added, translated, and deleted.

The first image processing part 24 has the function to perform necessary image processing, such as parallel translation processing, rotation processing, subtraction processing and projection processing, of X-ray image data generated in the image generation part 19 or the first image rotation part 21, according to instructing information from the first input device 16. The first image processing part 24 also has the function to display the X-ray image data after the image processing on the first display unit 17. For example, when a ROI as shown in FIG. 5 has been reset, an X-ray image in the ROI may be displayed on the first display unit 17 by image processing instead of the adjustment of the aperture 12.

Furthermore, an X-ray image can be translated according to translation instructing information input from the first input device 16 to the first image processing part 24, with reference to the mark displayed, on the screen of the first display unit 17, by the first mark display part 23. Specifically, a position of the aortic valve depicted horizontally or vertically on an X-ray image after the rotation can be matched with the position of the mark displayed at a fixed position, by operating the first input device 16. In this case, if the rotation angle of the X-ray image is not adjusted, the first image processing part 24 performs parallel translation processing of the X-ray image data based on a parallel distance input from the first input device 16.

On the other hand, without the operation of the first input device 16, the first image processing part 24 may be configured to translate an X-ray image automatically by data processing of the reference image data, depicting at least one of the aorta of the object O and a device. For example, an X-ray image can be translated automatically based on the positional relation between the fixed position of the mark displayed by the first mark display part 23 and a position of a balloon extracted by data processing of balloon image data.

Specifically, X-ray image date can be translated so as to match the mark like a frame line showing an outline of a balloon with the mark displayed at the fixed position of the screen or so as to match the center positions mutually between the marks. Note that, the first image processing part 24 or the first angle acquisition part 20 can display the mark like a frame line showing an outline of a balloon on the first display unit 17 based on a result of image processing.

When X-ray images have been rotated and displayed or the control information has been obtained for displaying and rotating the X-ray images, based on a result of data processing for balloon image data, the first image processing part 24 performs the parallel translation processing of X-ray image data. In this case, the rotational processing by the first image rotation part 21 and the parallel translation processing by the first image processing part 24 may be single translation processing of X-ray image data. In other word, the display with the rotation and the parallel translation of X-ray image data can be performed automatically by the first image rotation part 21 and the first image processing part 24 so that an outline of a balloon extracted from balloon image data matches with the mark displayed at the fixed position of the screen.

Furthermore, in the case where X-ray image data are translated in parallel based on the position information of the mark displayed at the fixed position of the screen, the first image processing part 24 can store the parallel distance and perform a parallel translation of other X-ray image data automatically based on the stored parallel distance. Specifically, based on the translation distance of the X-ray image, other X-ray images rotated and displayed after the X-ray image can be translated automatically.

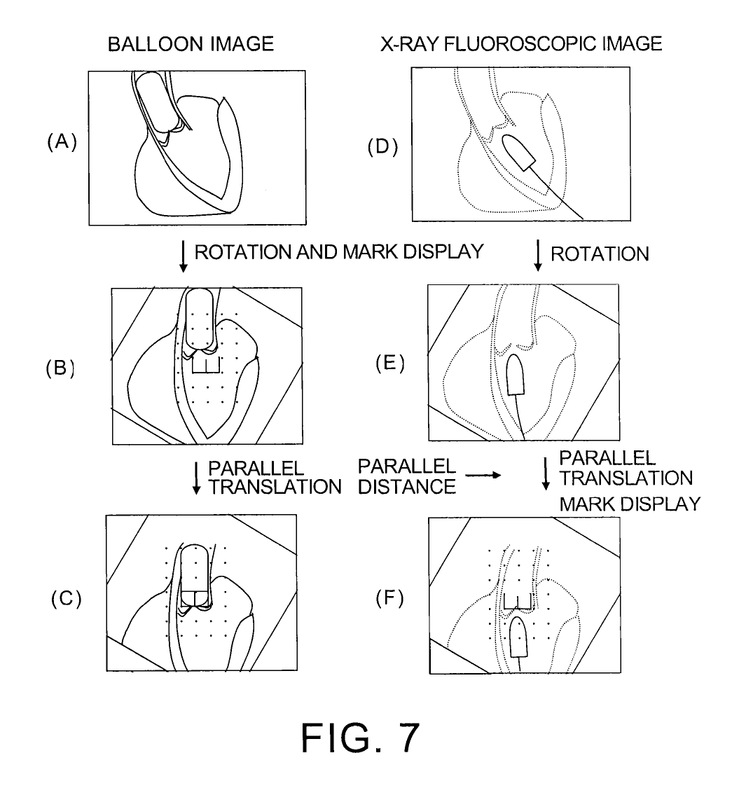

FIG. 7 is a diagram describing a method for translating an X-ray image using the marks displayed at fixed positions on the screen.

As shown in the FIG. 7 (A), in the case where balloon image data has been acquired as the reference image data, the inclined angle of the aorta can be detected automatically as an inclined angle of a balloon in the longitudinal direction with an outline of the balloon through the data processing of the balloon image data by the first angle acquisition part 20, as described above.

Next, a balloon image can be displayed with rotation, based on the inclined angle of the aorta, by the image processing by the first image rotation part 21, the rotation of the X-ray detector 8, the rotation of the object O, the rotation of the first display unit 17 or the rotational display of the screen of the first display unit 17. Consequently, a balloon image of which travel direction of the aorta is the horizontal or the vertical direction can be displayed as shown in the FIG. 7 (B).

Further, the mark to adjust the display position of the aortic valve can be displayed at the center of the screen by the first mark display part 23 as shown in the FIG. 7 (B). Additionally, the marks like the dot group to grasp a distance by the visual observation can be displayed as well. FIG. 7 (B) shows the example to display the mark, made by mutually connecting the respective ends of three parallel lines matched with the shape of the aortic valve with a vertical line, for the adjustment of the display position, together with the dot group for the grasp of a distance.

At this time, each of the marks for the distance confirmation and the mark for the display position adjustment displayed by the first mark display part 23 can be a mark corresponding to a width of the balloon image. In the example shown in FIG. 7 (B), the distance between the lines at the both ends for the display position adjustment is set to the width of the balloon. Furthermore, the pitch of the dot group for the distance confirmation is set as half the distance between the lines at the both ends for the display position adjustment and the width of the balloon. Therefore, it becomes possible to match the outline of the balloon with the mark for the display position adjustment by parallel translation of the balloon image data.

Accordingly, the balloon image can be translated in parallel through the parallel translation processing of the balloon image data by the first image processing part 24.so that the frame line showing the outline of the balloon overlaps with the lines at the both ends in the mark for the display position adjustment. Consequently, a balloon image, of which balloon is positioned at the mark displayed and fixed at the center of the screen, is displayed on the screen of the first display unit 17 as shown in the FIG. 7 (C).

Herewith, it becomes possible to confirm the position of the centerline of the balloon as the position of the mid line displayed at the center of the screen easily by visual observation. Additionally, it becomes possible to measure a distance in millimeter unit by visual observation with the use of the dot group for the grasp of the distance. Specifically, it becomes possible to visually measure the diameter of the aorta, the length of the aortic valve, the distance between the coronary artery and the aortic valve and the like.

If the aorta is displayed in an oblique direction as conventionally, the work to display a virtual ruler obliquely on a screen and to designate two dots on an image to measure a distance between the two dots is required. By contrast, it becomes possible to grasp various distances by visual observation without the operation of the first input device 16 by taking advantage of the mark for the distance measurement displayed in the horizontal direction or the vertical direction as shown in the FIG. 7 (C).

When balloon image data is translated in parallel as described above, the first image processing part 24 can store the translation distance so that the stored translation distance can be used for parallel translation processing of other X-ray images to be displayed later.

When the artificial valve is placed, X-ray fluoroscopic images depicting a device are displayed in real time. FIG. 7 (D) shows an aspect of an X-ray fluoroscopic image. However, a contrast agent is not injected in general before an acquisition of X-ray fluoroscopic images. Accordingly, blood vessels are not depicted on actual X-ray fluoroscopic images although the blood vessel is indicated by the dotted lines in FIG. 7 (D). That is, a user can observe a device visually through X-ray fluoroscopic images but not blood vessels.

However, X-ray fluoroscopic images can be rotated and displayed as shown in the FIG. 7 (E) by the image processing in the first image rotation part 21, the rotation of the X-ray detector 8, the rotation of the object O, the rotation of the first display unit 17 or the rotational display of the screen of the first display unit 17 since the inclined angle of the aorta has been detected in the first angle acquisition part 20.

Next, the first image processing part 24 translates the X-ray fluoroscopic image in parallel by the parallel distance of the balloon image. Consequently, an X-ray fluoroscopic image of which the center of the screen is the position of the aortic valve can be displayed as shown in the FIG. 7 (F). Further, the first mark display part 23 can display the mark for the display position adjustment and the dot group for the distance confirmation, according to the width of the balloon image, at the fixed position of the screen on which the X-ray fluoroscopic image after the parallel translation has been displayed.

Then, the mark for the display position adjustment shows the position of the aortic valve that cannot be observed visually on the X-ray fluoroscopic image. Furthermore, the mark for the distance confirmation can be used for the grasp of distances and parts that cannot be observed visually.

Accordingly, a user can use the mark for the display position adjustment as a mark for the positioning of a device like the artificial valve. Specifically, the accurate positioning of the artificial valve and devices can be performed by translating a device like the artificial valve to match the device with the mark. That is, it becomes possible to perform the accurate positioning of a device by using the mark though a user has conventionally memorized a position of the aortic valve on a reference image displayed before displaying X-ray fluoroscopic images to perform the positioning of a device roughly through the X-ray fluoroscopic images based on the memory.

Note that, FIG. 7 shows the example of translating the balloon image and the X-ray fluoroscopic image automatically in parallel to the mark by image processing using the balloon mage. However, a reference image other than a balloon image and an X-ray fluoroscopic image can be translated in parallel with reference to the reference image. In this case, the translation distance to translate the reference image to match the mark may be input from the first input device 16 to the first image processing part 24.

In other words, a reference image may be translated manually with reference to the mark by the operation of the first input device 16, and a translation distance of the reference image may be acquired and stored by the first image processing part 24. In this case, the first image processing part 24 translates other X-ray image data automatically in parallel based on the translation distance of the reference image acquired according to the information input from the first input device 16. That is, not by setting a region on a reference image, but by a translation of the reference image itself with referring to the mark for the positioning, a condition in translation processing can be set for other X-ray images.

Furthermore, although the above-mentioned explanation describes the example of displaying a reference image like a balloon image and X-ray fluoroscopic images with a parallel translation by image processing in the first image processing part 24, the display with the parallel translation of X-ray images can be also performed by translation of the X-ray detector 8 or the bed 10. In this case, the control information output part 22 acquires and stores a translation distance of a reference image, and outputs control information to the imaging position control unit 15 based on the stored translation distance of the reference image.

Specifically, in the case of calculating a translation distance of a reference image automatically, the control information output part 22 can calculate the translation distance of the reference image based on the result information of image processing, like an outline of a balloon acquired from the first angle acquisition part 20, and the position information of the mark for adjusting a display position of the aortic valve acquired from the first mark display part 23 On the other hand, in the case of determining the translation distance of the reference image manually by using the mark, the control information output part 22 can acquire the translation distance of the reference image from the first input device 16.

Then, as X-ray imaging is performed at the position translated by the translation distance of the reference image, the control information output part 22 can output the control information to the imaging position control unit 15. That is, an imaging region of other X-ray images can be set by using the reference image as an image for positioning of X-ray imaging, in addition to not by setting the imaging region on the image for positioning but by translating the image itself for positioning with reference to the mark for positioning.

The first image memory 25 is a storage unit to store X-ray image data generated by the image generation part 19, the first image rotation part 21, the first image processing part 24 or the like.

On the other hand, the second screen rotation part 31, the second angle acquisition part 32, the second image rotation part 33, the second mark display part 34, the second image processing part 35, the second image memory 36 and the second inclined angle storage unit 37 included in the medical image processing apparatus 3 have functions similar to those of the first screen rotation part 18, the first angle acquisition part 20, the first image rotation part 21, the first mark display part 23, the first image processing part 24, the first image memory 25 and the first inclined angle storage unit 26 included in the X-ray imaging apparatus 1 respectively. The second automatic detecting part 38 and the second manual instructing part 39 included in the second angle acquisition part 32 of the medical image processing apparatus 3 also have functions equivalent to those of the first automatic detecting pan 27 and the first manual instructing part 28 included in the first angle acquisition part 20 of the X-ray imaging apparatus 1 respectively. Accordingly, the medical image processing apparatus 3 can also generate X-ray image data similar to that by the data processing system 6 of the X-ray imaging apparatus 1 and display the generated X-ray image data on the second display unit 30.

Then, the X-ray imaging apparatus 1 has a function as an imaging unit to display an X-ray image with rotating the angle so as to make a travel direction of the aorta to be the horizontal direction or the vertical direction based on an inclined angle of the aorta of an object acquired with reference to a reference image, by cooperative working of the elements included in the X-ray imaging apparatus 1. Similarly, the medical image processing apparatus 3 has a function as a display control unit to display an X-ray image with rotating the angle so as to make a travel direction of the aorta to be the horizontal direction or the vertical direction based on an inclined angle of the aorta of an object acquired with reference to a reference image, by cooperative working of the elements included in the medical image processing apparatus 3. However, so long as equivalent functions are provided in the X-ray imaging apparatus 1 and the medical image processing apparatus 3, the X-ray imaging apparatus 1 and the medical image processing apparatus 3 can be configured by elements other than the elements exemplified in FIG. 1.

Next, the operation and action of the X-ray imaging apparatus 1 and the medical image processing apparatus 3 will be described.

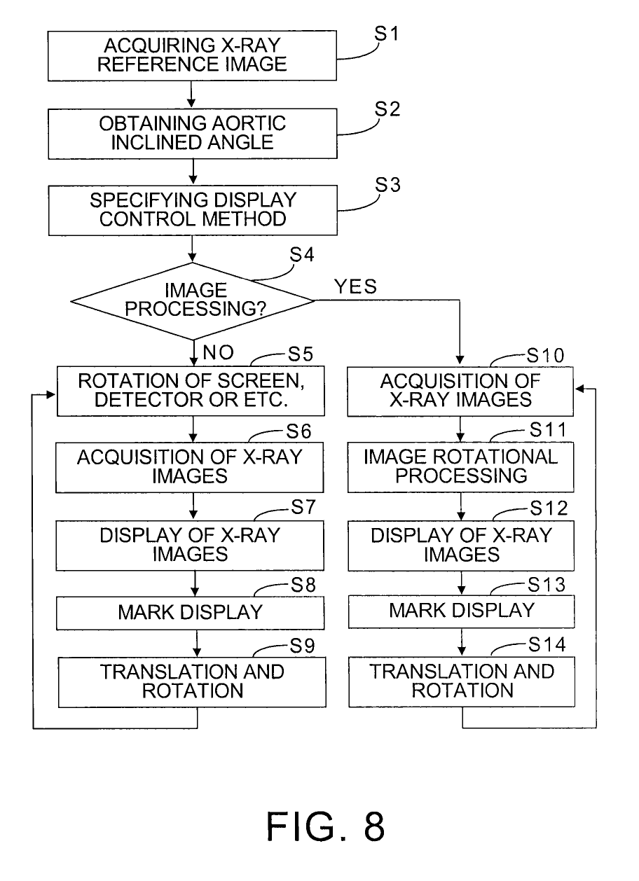

FIG. 8 is a flowchart showing a flow for displaying X-ray images for a replacement of an aortic valve of an object O by the X-ray imaging apparatus 1 shown in FIG. 1.

Firstly, in the step S1, X-ray images are obtained for reference by the X-ray imaging apparatus 1. Specifically, an object is set on the bed 10, and subsequently the detector driving mechanism 9 and the bed driving mechanism 11 are driven according to control information from the imaging position control unit 15. Then, the bed 10, the X-ray exposure unit 7 and the X-ray detector 8 are positioned at predetermined spatial positions with predetermined rotation angles. Furthermore, the opening of the aperture 12 is adjusted under the control by the aperture control unit 14.

Then, an X-ray is exposed to the object O from the X-ray tube of the X-ray exposure unit 7 by applying a high voltage to the X-ray tube from the high voltage generator 13. The X-ray transmitting the object O is detected by the X-ray detector 8.

The X-ray detection data acquired by the X-ray detector 8 is output to the data processing system 6. Then, the image generation part 19 generates X-ray image data by data processing of the X-ray detection data. The generated X-ray image data is displayed on the first display unit 17. Herewith, it becomes possible for a user to refer to the X-ray image.

Furthermore, a catheter is inserted in a blood vessel of the object O. Therefore, X-ray fluoroscopic images depicting the catheter can be displayed on the first display unit 17 in real time. Further, devices such as a balloon and/or a wire are inserted in the aorta branched from the heart through the catheter, as needed. Herewith, balloon images and/or wire images are displayed on the first display unit 17 in real time.

Furthermore, blood vessel contrast-enhanced X-ray images are generally obtained. In this case, a contrast agent is injected in the object O from the contrast agent injector 40 before the imaging. However, there is a case where contrast-enhanced images are not obtained.

Then, among the frames of the X-ray image data acquired as described above, frames of X-ray image data referred to for obtaining an inclined angle of the aorta are stored in the first image memory 25.

Next, in the step S2, the first angle acquisition part 20 acquires the inclined angle of the aorta of the object O on a reference image by using X-ray image data depicting the aorta of the object O or a device as reference image data. For example, if X-ray image data depicting a landmark showing the inclined angle of the aorta like balloon image data is used as the reference image data, the first automatic detecting part 27 detects the angle of the aorta automatically by image processing of the reference image data

On the other hand, if X-ray image data depicting no landmarks showing the inclined angle of the aorta is used as the reference image data, the first manual instructing part 28 displays a reference image such as an X-ray contrast-enhanced image or an X-ray fluoroscopic image on the first display unit 17 with overlapping a graphic symbol, for instructing the inclined angle of the aorta, on the reference image. When a user rotates the graphic symbol, for instructing the inclined angle of the aorta, to match the graphic symbol with a direction of the aorta by operating the first input device 16, the rotation angle of the graphic symbol is input from the first input device 16 to the first manual instructing part 28. Herewith, the first manual instructing part 28 can acquire the inclined angle of the aorta based on the rotation angle of the graphic symbol.

Furthermore, the first automatic detecting part 27 displays a graphic symbol, showing the automatically detected inclined angle of the aorta, on the reference image, as needed. Then, the user can correct the inclined angle of the aorta, detected automatically by the first automatic detecting part 27, by inputting rotational information of the graphic symbol from the first input device 16 to the first manual instructing part 28.

Thus, the inclined angle of the aorta that is confirmed finally is stored in the first inclined angle storage unit 26. Therefore, it is possible to confirm the inclined angle of the aorta when it is needed. Furthermore, it becomes possible to perform a display control based on the inclined angle of the aorta so that X-ray images, of which travel direction of the aorta is directed toward the horizontal or vertical direction, is displayed on the first display unit 17.

Next, in the step S3, a method of the display control is designated by the operation of the first input device 16. The method by image processing, the method by rotating the X-ray detector 8 or the object O and the method by rotating the display screen of the first display unit 17 are selectable as the method of the display control. Specifically, the select information to select one of the above-mentioned three methods is input to the data processing system 6 by the operation of the first input device 16.

Next, in the step S4, which method has been designated for the display control method is determined by the data processing system 6. For example, it is determined whether the image processing has been selected as the method of the display control or not. Note that, without the actual determination processing by the data processing system 6, each element of the data processing system 6 may simply respond to the information input from the first input device 16.

Then, when it is determined that the image processing has not been selected as the method of the display control, a target designated by the operation of the first input device 16 is rotated, in the step S5.

Specifically, when the display screen of the first display unit 17 has been designated as the rotational target, the control information output part 22 controls the first screen rotation part 18 based on the inclined angle of the aorta so that the angle of the travel direction of the aorta becomes the horizontal or vertical direction in the X-ray image displayed on the first display unit 17. Therefore, the first screen rotation part 18 inclines the screen angle of the first display unit 17 so that the incline angle of the aorta displayed on the screen of the first display unit 17 becomes the horizontal or vertical direction.

On the other hand, when the X-ray detector 8 or the object O has been designated as the rotational target, the control information output part 22 controls the imaging position control unit 15 based on the inclined angle of the aorta so that the angle of the travel direction of the aorta becomes the horizontal or vertical direction in the X-ray image displayed on the first display unit 17. Therefore, the imaging position control unit 15 drives the detector driving mechanism 9 or the bed driving mechanism 11 to adjusts the rotation angle of the X-ray detector 8 and the X-ray exposure unit 7 or the inclined angle of the table of the bed 10 so that the inclined angle of the aorta becomes the horizontal or vertical direction in the X-ray image displayed on the first display unit 17.

Subsequently, in the step S6, X-ray images are acquired in a flow similar to that in the step S1, in the state where the rotation angle of the X-ray exposure unit 7 and the X-ray detector 8, or the inclined angle of the table of the bed 10 has been adjusted. Furthermore, the user can proceed with the artificial valve in the vicinity of the aortic valve through the catheter.

Next, in the step S7, the image generation part 19 displays the acquired X-ray images on the first display unit 17 in real time. Alternatively, the first image processing part 24 performs necessary image processing, such as subtraction processing or projection processing, of the X-ray image data acquired from the image generation part 19 and displays the processed X-ray image data on the display unit 17 in real time.

At this time, X-ray images of which the travel direction of the aorta is the horizontal or vertical direction are displayed on the first display unit 17 since the rotation angle of the X-ray exposure unit 7 and the X-ray detector 8, the inclined angle of the table of the bed 10 or the inclined angle of the screen of the first display unit 17 has been adjusted so that the travel direction of the aorta in the X-ray images is not inclined.

Next, in the step S8, the first mark display part 23 overlaps and displays marks, such as dots, lines, a grid, and a cross symbol as shown in FIG. 6, at desired positions on the X-ray images rotated and displayed on the first display unit 17. Herewith, the user can perform positioning including an angle adjustment of the artificial valve with confirming the horizontal direction and the vertical direction as well as distances.

Next, in the step S9, the user can translate or rotate an X-ray image displayed on the screen of the first display unit 17 by the operation of the first input device 16, as needed. Specifically, when rotational instruction or parallel translation instruction of an X-ray image is input from the first input device 16 to the first image processing part 24, the first image processing part 24 performs the rotational processing or the parallel translation processing of X-ray image data acquired from the image generation part 19 and displays the processed X-ray image data on the display unit 17 in real time.

It is necessary for positioning of the artificial valve to move the artificial valve to a target position in addition to the adjustment in the angle of the artificial valve. Accordingly, a whole X-ray image can be translated in parallel so that a mark like the cross symbol overlapped and displayed on the X-ray image lies on a target position of the aortic valve or the like, or a reference position corresponding to the target position. For example, a whole X-ray image can be translated in parallel so that a crossed mark displayed at the screen center of the first input device 16 is positioned at the lower left of the aorta.

Alternatively, as shown in FIGS. 7 (B) and (C), a whole X-ray image can be automatically translated in parallel so that a position of the balloon detected from the balloon image data matches with the mark displayed at the fixed position, like the screen center of the first input device 16, by the first mark display part 23. Note that, a translation of the whole X-ray image can be performed not only by image processing of the first image processing part 24 but also by a translation of the X-ray detector 8 or the bed 10 by the control information output part 22 through the imaging position control unit 15.

Such a translation of an X-ray image makes it possible to perform the positioning of the artificial valve easily and accurately by moving the artificial valve with targeting a mark displayed at a fixed position of the screen.

Then, when the artificial valve is placed, X-ray images such as X-ray fluoroscopic images, DSA images or road map images used for reference images can be acquired and displayed in real time. In the case where correction information of the inclined angle of the aorta is input from the first input device 16 to the first angle acquisition part 20 as a rotation instruction, the X-ray exposure unit 7 and the X-ray detector 8, the table of the bed 10 or the inclined angle of the screen of the first display unit 17 is adjusted again in the step S5. Then, X-ray images are acquired and displayed in real time again.

When the artificial valve is placed, X-ray fluoroscopic images are acquired and displayed in real time without injecting a contrast agent typically. In this case, devices including the artificial valve are depicted but the aorta is not depicted clearly. Accordingly, as shown in the FIG. 7 (F), the X-ray fluoroscopic images can be displayed with an automatic translation based on a parallel distance of the X-ray image used as the reference image for the acquisition of the angle of the aorta. Additionally, the mark to grasp a position of the aortic valve, at the fixed position of the screen, for the positioning of the artificial valve and the marks for distance confirmation can be displayed.

Accordingly, after the rotational angle and the parallel distance of the X-ray images are fixed once, the translation of the X-ray detector 8 or the bed 10 in the step S5 and the step S9 can be omitted. However, in case of performing the parallel translation of the X-ray fluoroscopic images by image processing, the parallel translation processing of the X-ray fluoroscopic image data is performed in the first image processing part 24 before the display of the X-ray images in the step S7. Furthermore, each display of the step S7 and the step S8 may be performed simultaneously or in the reverse order.

On the other hand, if it is determined that the image processing has been selected as the method of the display control in the step S4, X-ray image data used as reference images for placing the artificial valve is acquired in the step S10 in a flow similar to that in the step S6.

Next, in the step S11, the first image rotation part 21 performs rotational processing of the X-ray image data based on the inclined angle of the aorta, so that the angle of the travel direction of the aorta in X-ray images displayed on the first display unit 17 becomes the horizontal or vertical direction. Specifically, positions of respective pixels of the X-ray image data are rotated reversely by the inclined angle of the aorta. Herewith, the X-ray image data of which travel direction of the aorta is the horizontal or vertical direction is generated.

Next, in the step S12, the first image rotation part 21 displays the X-ray image data after the rotational processing on the first display unit 17. After that, in the step S13 and the step S14, the display of the marks and rotation or parallel translation of the X-ray images can be performed similarly to the step S8 and the step S9,

Further, the works including the acquisition and the display of X-ray images from the step S10 to the step S14 are repeated in real time for the positioning of the artificial valve. Typically, non-contrast-enhanced X-ray fluoroscopic images are displayed in real time. In this case, the translation of the X-ray detector 8 or the bed 10 can be also omitted after a parallel distance of the X-ray images is fixed once. Furthermore, in case of performing the parallel translation of the X-ray fluoroscopic images by image processing, the parallel translation processing of the X-ray fluoroscopic image data is performed before the display of the X-ray images. Furthermore, each display of the X-ray images and the marks in the step S12 and the step S13 can be performed simultaneously or in the reverse order.

Note that, a ROI can be set again in the case where unnecessary regions are displayed on the corners of the screen of the first display unit 17 as a result of the display control of X-ray images. Then, X-ray images in the ROI can be enlarged and displayed. In this case, X-ray detection data only in the ROI may be acquired under the control of the aperture control unit 14 by the control information output part 22.

Then, when the placement of the artificial valve is completed, the X-ray images can be displayed on the first display unit 17 at the actual display angle. Specifically, in the case where the instruction ending the rotational display mode of X-ray images is input to the data processing system 6 by the operation of the first input device 16, the control information output part 22 outputs the instruction to turn the inclined angle back as a control signal to the first screen rotation part 18 or the imaging position control unit 15. Alternatively, the first image rotation part 21 stops the image processing so that the X-ray image data is output from the image generation part 19 or the first image processing part 24 to the first display unit 17 at the actual inclined angle. Herewith, X-ray images of the actual inclined angle with the inclining aorta are displayed on the first display unit 17.

Note that, with regard to the medical image processing apparatus 3, X-ray images can be displayed with a rotation and a parallel translation in a similar flow. Accordingly, the description for the flow in the medical image processing apparatus 3 is omitted.

That is, the above-mentioned X-ray imaging apparatus 1 and medical image processing apparatus 3 are apparatuses configured to acquire an inclined angle of the aorta based on a reference image to display X-ray images with a rotation so that the aorta is displayed in the vertical or horizontal direction based on the acquired inclined angle.

Therefore, according to the X-ray imaging apparatus 1 and the medical image processing apparatus 3, it is possible to place an artificial valve in the horizontal or vertical direction on an X-ray image referred to in the replacement technique of aortic valve. Specifically, a user who is a doctor can perform the positioning of the artificial valve with satisfactory accuracy and no difficulty by a rotation and a parallel translation of an object in the horizontal or vertical direction which are easy to be recognized.

Especially, according to the X-ray imaging apparatus 1, not only rotational display processing of X-ray images but useful display processing for display with a parallel translation can be performed. Therefore, a user can devote a craft for placing the artificial valve so long as the user performs at least a parallel translation of a displayed X-ray image. Specifically, it becomes possible to make works, such as drawing lines and marking by operating an input device with reference to an X-ray image, unnecessary.

On the other hand, although a parallel translation of X-ray images can be performed by moving the X-ray detector 8 or the bed 10, a movement of the X-ray detector 8 or the bed 10 is performed frequently. Accordingly, it is possible for the user to perform a parallel translation of X-ray images as a practiced operation. Therefore, works obstructing a craft can be reduced.

Further, if a parallel translation of displayed X-ray images is also performed automatically by image processing of a reference image like a balloon image, it is possible to further reduce works of a user. Therefore, the work for placing the artificial valve can proceed without any problems.

### Second Embodiment

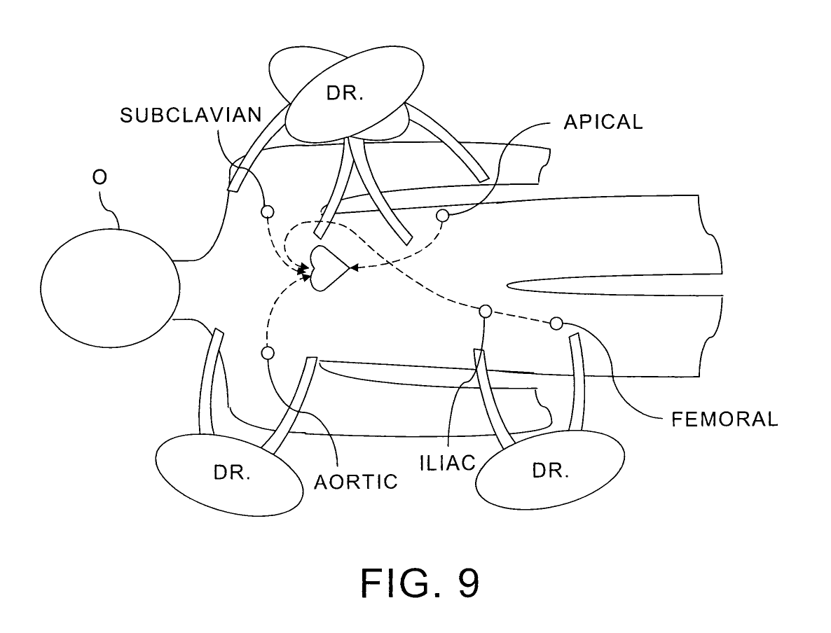

FIG. 9 is a view for explaining functions of an X-ray imaging apparatus and a medical image processing apparatus according to the second embodiment of the present invention.

The X-ray imaging apparatus and the medical image processing apparatus in the second embodiment are different from the X-ray imaging apparatus 1 and the medical image processing apparatus 3 in the first embodiment in a point that X-ray images are rotated and displayed so as to make an aorta displayed in a predetermined angle based on an inclined angle of the aorta acquired from a reference image. The other constructions and operations of the X-ray imaging apparatus and the medical image processing apparatus in the second embodiment are not different from those of the X-ray imaging apparatus 1 and the medical image processing apparatus 3 in the first embodiment substantially. Therefore, only a determination method of a display angle of an aorta will be described.

FIG. 9 shows respective standing positions of a doctor against an object O for craft methods in TAVI. In TAVI, a user who is a doctor stands at a position according to a craft and inserts a device such as an artificial valve and/or a balloon in a blood vessel of an object O to advance the device toward a placed position of the artificial valve. Accordingly, a travel direction of the aorta as viewed from a user varies depending on a craft and becomes a direction corresponding to the craft.

The known craft methods adopted for the TAVI include the subclavian method which inserts an artificial valve into an object O from the subclavian artery, the aortic method which inserts an artificial valve from the aorta, the apical method which inserts an artificial valve from the cardiac apex, the femoral method which inserts an artificial valve from the femur, and the iliac method which inserts an artificial valve from the iliac, as shown in FIG. 9.

Accordingly, X-ray images can be displayed with rotating the angle so as to make a travel direction of the aorta become a predetermined direction corresponding to a craft based on an inclined angle of the aorta acquired with reference to a reference image. Specifically, X-ray images can be rotated and displayed so that an inclined angle of the aorta acquired with reference to a reference image becomes the travel direction of the aorta, at a placed position of the artificial valve, as viewed from a user.

Specifically, the travel directions of the aorta as viewed from the craft positions corresponding to one or multiple crafts, which can be adopted in the TAVI, are stored in a storage unit of at least one of the X-ray imaging apparatus and the medical image processing apparatus. Specifically, the angles of the travel directions of the aorta corresponding to the respective crafts are preset as reference directions. When information specifying a craft by an operation of an input device is input to the X-ray imaging apparatus or the medical image processing apparatus, the X-ray imaging apparatus or the medical image processing apparatus acquires a travel direction of the aorta, as viewed from the craft position corresponding to the specific information of the craft, by referring to the storage unit. The acquired travel direction of the aorta as viewed from the craft position is determined as the predetermined direction serving as a reference for determining a rotation angle of X-ray images.

Then, X-ray images are rotated and displayed so as to make the travel direction of the aorta become the direction corresponding to the specified craft. If the control target is the medical image processing apparatus, a rotation of a screen or image rotational processing can be adopted as the rotational display method of X-ray images, similarly to the first embodiment. On the other hand, if the control target is the X-ray imaging apparatus, a rotation of a screen, a rotation of the X-ray detector, or image rotational processing can be adopted.

As described above, the X-ray imaging apparatus has a function as an imaging unit to display X-ray images with rotating the angle based on an inclined angle of the aorta of an object acquired with reference to a reference image so that the travel direction of the aorta becomes a predetermined direction corresponding to a craft. Similarly, the medical image processing apparatus has a function as a display control unit to display X-ray images with rotating the angle based on an inclined angle of the aorta of an object acquired with reference to a reference image so that the travel direction of the aorta becomes a predetermined direction corresponding to a craft. The imaging unit and the display control unit can be configured by arbitrary elements as exemplified by FIG. 1.

That is, the X-ray imaging apparatus and the medical image processing apparatus in the second embodiment are apparatuses configured to display X-ray images with a rotation so that the travel direction of the aorta becomes a relative position of a user with respect to an object O and the angle corresponding to a craft method instead of displaying and rotating the X-ray images so that the travel direction of the aorta becomes the horizontal or vertical direction.

Therefore, a travel direction of the aorta which is a sending direction of an artificial valve in the vicinity of the placed position of the artificial valve as viewed from a user can be equivalent to a travel direction of the artificial valve depicted on X-ray images. Then, a user can perform a craft with observing X-ray images depicting a device such as the artificial valve that moves toward a same direction as an actual sending direction.

### Other Embodiments

While certain embodiments have been described, these embodiments have been presented by way of example only, and are not intended to limit the scope of the inventions. Indeed, the novel methods and systems described herein may be embodied in a variety of other forms; furthermore, various omissions, replacement and changes in the form of the methods and systems described herein may be made without departing from the spirit of the inventions. The accompanying claims and their equivalents are intended to cover such forms or modifications as would fall within the scope and spirit of the inventions.

For example, not only for display of X-ray images for a replacement of an aortic valve but for display of X-ray images for a treatment such as a replacement of a mitral valve, a penetrating treatment of a valve occluded completely, a centesis, and an aortic graft, displayed images can be rotated so that an oblique landmark becomes horizontal, vertical or a predetermined angle on a screen. In a case of a treatment like a centesis, non-contrast-enhanced blood vessel images may be display targets. Furthermore, when any other than a blood vessel is a display target, similar display processing or display control of images can be also performed.

Further, in the case of real time imaging by using another image diagnostic apparatus such as an X-ray CT apparatus, an MRI apparatus or an ultrasonic diagnostic apparatus, similar display processing or display control of images can be also performed. Especially, in the case of imaging with focusing on specified slices, an application of the above described display processing or display control of images can be expected.

What is claimed is:

1. An X-ray imaging apparatus comprising:
an angle acquisition unit configured to acquire an inclined angle of an aorta of an object on a reference image; and
an imaging unit configured to display an X-ray image with rotating an angle so as to make a travel direction of the aorta to be a horizontal direction, a vertical direction or a predetermined direction corresponding to a craft, the angle being rotated based on the inclined angle of the aorta.

2. An X-ray imaging apparatus of claim 1,
wherein said angle acquisition unit is configured to automatically detect the inclined angle of the aorta by data processing of balloon image data depicting a balloon inserted into the aorta.

3. An X-ray imaging apparatus of claim 1,
wherein said angle acquisition unit is configured to display and overlap a figure, for instructing an inclined angle, on the reference image to acquire the inclined angle of the aorta based on a rotational angle of the figure input from an input device.

4. An X-ray imaging apparatus of claim 1,
wherein said imaging unit is configured to display the X-ray image by inclining at least one of an X-ray detector and the object based on the inclined angle of the aorta.

5. An X-ray imaging apparatus of claim 1,
wherein said imaging unit is configured to display the X-ray image by performing image processing of X-ray imaging data of the aorta based on the inclined angle of the aorta.

6. An X-ray imaging apparatus of claim 1,
wherein said imaging unit is configured to display the X-ray image by inclining a display screen for displaying the X-ray image, based on the inclined angle of the aorta.

7. An X-ray imaging apparatus of claim 1, further comprising:
a mark display unit configured to display a mark at a fixed position on a screen of a display unit displaying the X-ray image, a shape of the mark being adjusted based on a shape of a balloon inserted into the aorta, the shape of the balloon being extracted by data processing of balloon image data depicting the balloon.

8. An X-ray imaging apparatus of claim 1,
wherein said imaging unit is configured to automatically translate the X-ray image based on a positional relation between a position of a balloon extracted by data processing of balloon image data depicting the balloon and a mark displayed at a fixed position on a screen of a display unit displaying the X-ray image, the balloon being inserted into the aorta.

9. An X-ray imaging apparatus of claim 8,
wherein said imaging unit is configured to automatically translate another X-ray image rotated to be displayed after the X-ray image, based on a translation distance of the X-ray image.

10. An X-ray imaging apparatus of claim 1,
wherein said imaging unit is configured to set an imaging region or a translation processing condition of another X-ray image by a translation of the X-ray image with referring to a mark displayed at a fixed position on a screen of a display unit displaying the X-ray image.

11. An X-ray imaging apparatus of claim 1, further comprising:
a storage unit configured to store traveling directions of aortas as viewed from craft positions corresponding to crafts which may be adopted for a trans-catheter aortic valve replacement,
wherein said imaging unit is configured to acquire a traveling direction of the aorta as viewed from a craft position corresponding to information specifying the craft by referring to the storage unit, the traveling direction of the aorta as viewed from the craft position being acquired as the predetermined direction.

12. A medical image processing apparatus comprising:
an angle acquisition unit configured to acquire an inclined angle of an aorta of an object on a reference image; and
a display control unit configured to display an X-ray image with rotating an angle so as to make a travel direction of the aorta to be a horizontal direction, a vertical direction or a predetermined direction corresponding to a craft, the angle being rotated based on the inclined angle of the aorta.

13. An X-ray imaging method comprising:
acquiring an inclined angle of an aorta of an object on a reference image; and
displaying an X-ray image with rotating an angle so as to make a travel direction of the aorta to be a horizontal direction, a vertical direction or a predetermined direction corresponding to a craft, the angle being rotated based on the inclined angle of the aorta.
